(19) United States
(12) Patent Application Publication
(10) Pub. No.: US 2026/0363615 A1
(43) Pub. Date: Nov. 19, 2026

(54) PSYCHOPHARMACOLOGICAL SYSTEM AND METHOD USING EYELID TRACKING

(71) Applicant: BLINKLAB, Princeton, NJ (US)

(72) Inventors: Sebastiaan Karel Emiel KOEKKOEK, Princeton, NJ (US); Henk-Jan BOELE, Princeton, NJ (US); Anton UVAROV, Princeton, NJ (US); Peter BOELE, Princeton, NJ (US)

(73) Assignee: BLINKLAB, Princeton, NJ (US)

(21) Appl. No.: 19/133,953

(22) PCT Filed: Nov. 30, 2023
(86) PCT No.: PCT/US2023/081810

§ 371 Date: May 29, 2025

Related U.S. Application Data
(60) Provisional application No. 63/428,952, filed on Nov. 30, 2022.

(51) Int. Cl. A61B 5/00; A61B 3/14; A61B 5/11; A61B 5/16; G06T 7/00; G16H 20/10

(52) U.S. Cl. CPC A61B 5/4848; A61B 3/14; A61B 5/1103; A61B 5/1128; A61B 5/163; A61B 5/165; A61B 5/7246; G06T 7/0012; G16H 20/10; G06T 2207/30041

(57) ABSTRACT

A technique for identifying correct or sufficient dosing in patients being treated with a medication for neurodeviate conditions is provided. The method may include performing multiple tests of a startle response of a user, each test utilizing a device having a camera, display, and optionally a speaker, and each test occurring a different time after the user has been administered a medication. The method may include receiving a plurality of images of at least one eye of the user from a camera during each test, then calculating amplitudes of eyelid closure based on each image. The method may include determining a value of a correlation between predetermined plasma concentrations of the medication and the determined amplitudes, and then determining whether a correct or sufficient dose has been achieved based on the value of the correlation.

### CROSS-REFERENCE TO RELATED APPLICATIONS

The present application claims priority to U.S. Provisional Patent Application No. 63/428,952, filed Nov. 30, 2022, the contents of which are incorporated by reference herein in its entirety.

### TECHNICAL FIELD

The present disclosure is drawn to psychopharmacology, and specifically to using computer-based eyelid-tracking technology to assist in identifying correct medication dosing for patients with neurodeviate conditions, such as autism spectrum disorder, ADHD, and schizophrenia.

### BACKGROUND

Psychopharmacology is the study of the use of medications in treating mental disorders. The complexity of this field requires psychopharmacologists to understand, inter alia, all the clinically relevant principles of pharmacokinetics and pharmacodynamics.

When identifying a treatment plan, a psychopharmacologist will typically have access to a wide range of literature, clinical studies, and data (such as blood plasma concentrations) showing how long a drug will be effective in a person's system. However, even with all that information, when a patient with a neurological disease is treated using medication, a psychopharmacologist may take upwards of six months, with much trial and error, to find the correct dose for a particular patient.

### BRIEF SUMMARY

Various deficiencies in the prior art are addressed below by the disclosed techniques and systems. The disclosed systems and methods may be used to, e.g., reduce the time required to identify whether a prescribed dosage is correct.

In some embodiments, a method for identifying correct dosing in patients being treated with a medication for neurodeviate conditions may be provided. The method may include performing three of more tests of a startle response of a user, each test occurring a different time after the user has been administered a medication. In preferred embodiments, each test may utilize a mobile device having a camera, display, and optionally a speaker. The method may include receiving a plurality of images of at least one eye of the user from a camera during each test. The method may include calculating amplitudes of a closure of an eyelid of the at least one eye for each test. The method may include determining a value of a correlation between predetermined plasma concentrations of the medication and the amplitudes of the closure of the eyelid at the different times after the user has been administered the medication. The method may include determining whether a correct dose has been achieved based on the value of the correlation. In some embodiments, all of these steps may be performed on the mobile device. In some embodiments, the mobile device may send the plurality of images to a remote processor, the remote processor being configured to calculate the amplitudes, determine the correlation, and determining whether the correct dose has been achieved.

In some embodiments, the amplitude may be positively correlated with the plasma concentrations of the medication. In some embodiments, the amplitude may be negatively correlated with the plasma concentrations of the medication.

The method may include recommending a modified dosage of the medication based on the correlation. In some embodiments, the method may include repeating the steps at the same medication dosage. In some embodiments, the method may include adjusting a dosage of the medication and repeating the steps. In some embodiments, the method may include receiving input indicating when the medication was administered. In some embodiments, the method may include receiving input indicating what dosage was administered. In some embodiments, the method may include storing information in a database, the information including a user code, a dose, a time of administration, and the amplitudes and times each of the plurality of images was captured.

In some embodiments, a system may be provided. The system may include one or more processors. The system may include a display operably coupled to a first processor of the one or more processors. The system may include a camera operably coupled to the first processor. The system may include a speaker coupled to the first processor. The system may include a non-transitory computer-readable medium. The storage medium may contain instructions that, when executed, configure the one or more processors to, either individually or collectively, perform specific tasks. The processor(s) may be configured to cause the system to perform the method as disclosed herein. The processor(s) may be configured to perform three of more tests of a startle response of a user, each test utilizing the display, the speaker, or both, and each test occurring a different time after the user has been administered a medication. The processor(s) may be configured to receive a plurality of images of at least one eye of the user from the camera during each test. The processor(s) may be configured to calculate amplitudes of a closure of an eyelid of the at least one eye for each test. The processor(s) may be configured to determine a correlation between predetermined plasma concentrations of the medication with a curve formed by the amplitudes of the closure of the eyelid at the different times after the user has been administered the medication. The processor(s) may be configured to determine whether a correct dose has been achieved based on the value of the correlation.

In some embodiments, the first processor may be present on a mobile device. In some embodiments, all steps may be performed on the mobile device (e.g., by the first processor). In some embodiments, the first processor may present on a mobile device, a second processor of the one or more processors may be present on a remote device, and the first processor may be configured to send the plurality of images to the second processor. The second processor may be configured to calculate the amplitudes, determine the correlation, and determining whether the correct dose has been achieved. In some embodiments, the amplitude may be positively correlated with the plasma concentrations of the medication. In some embodiments, the amplitude is negatively correlated with the plasma concentrations of the medication.

The one or more processors may be configured to recommend a modified dosage of the medication based on the correlation. The one or more processors may be configured to receive input indicating when the medication was administered. The one or more processors may be configured to receive input indicating what dosage was administered. The one or more processors may be configured to store information in a database, the information including a user code, a dose, a time of administration, and the amplitudes and times each of the plurality of images was captured.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a flowchart of a method.

FIG. 2 is a simplified block diagram of a system.

FIG. 3 is a graphical depiction of testing startle responses and determining amplitudes of eyelid closure for each test for methylphenidate used at a 10 mg dosage.

FIG. 4 is a graph showing clinical data for plasma methylphenidate concentrations for two different pharmaceutical drugs at two different dosages.

FIG. 5 is a graph showing amplitudes of eyelid closure and plasma concentrations as measured across a period of time.

FIG. 6 is an illustration of a template for tracking facial landmarks, and eye landmarks in particular.

FIGS. 7A-7C are graphs showing eyelid closures over time after a prepulse that starts at time t=0, including N=9 matched controls for neurotypical individuals (7A), and N=10 individuals with ADHD before (7B) and after (7C) use of methylphenidate.

FIG. 8 is an illustration of neurocircuitries underlying auditory startle reflexes. The neurocircuits include: auditory cortex (AC); central amygdala (CE); cochlear nucleus (CN); cochlear root nucleus (CrN); dorsal cochlear nucleus (DCN); facial nucleus (FN); lateral amygdala (LA); lateral superior olive (LSO); medial geniculate body (MGB) of the thalamus; motor neurons (MN); caudal pontine reticular nucleus (PnC); ventral cochlear nucleus (VCN); and ventrolateral tegmental nucleus (VTN).

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

Surprisingly, it has been determined that for many medications used to treat neurological conditions, the eyeblink characteristics (such as amplitude of eyeblinks) can be used as a proxy for plasma medication concentrations.

Such medications may be a stimulant, such as methylphenidate, methylphenidate salts, amphetamines, amphetamine salts, and/or atomoxetine HCl. The medications may be an antidepressant and/or anxiolytic. The medications may be an amphetamine, a selective serotonin reuptake inhibitor (SSRI), or a psychedelic.

Amphetamines, such as methylphenidate, act by increasing the release and/or inhibiting the reuptake of neurotransmitters, particularly dopamine and norepinephrine, in the brain. Consequently, the concentration of these neurotransmitters in the synaptic cleft increases, leading to enhanced neurotransmission. This heightened neurotransmission is associated with increased alertness, elevated mood, improved focus, and a heightened sense of energy. The increased levels of dopamine and/or norepinephrine in brainstem circuits can be quantified using a simple brainstem reflex, namely the eyeblink startle reflex, and prepulse inhibition and habituation of this reflex.

Selective Serotonin Reuptake Inhibitors (SSRIs) function by inhibiting the reuptake of serotonin in the synaptic cleft, resulting in elevated levels of serotonin in the synaptic cleft. Serotonin is a neurotransmitter that plays a key role in mood regulation, among other functions. In a normally functioning synapse, after serotonin is released from the presynaptic neuron, it binds to receptors on the postsynaptic neuron. The increased levels of serotonin in brainstem circuits can be quantified using a simple brainstem reflex, namely the eyeblink startle reflex, and prepulse inhibition and habituation of this reflex.

Psychedelics, such as psilocybin (found in certain mushrooms), LSD (lysergic acid diethylamide), and DMT (dimethyltryptamine), exert their effects primarily through interactions with the serotonin system in the brain. The serotonin receptor subtype 5-HT2A is particularly implicated in the effects of psychedelics. The activation of 5-HT2A receptors leads to an increase in serotonin transmission in certain brain circuits, including the brainstem. The increased levels of serotonin in brainstem circuits can be quantified using a simple brainstem reflex, namely the eyeblink startle reflex, and prepulse inhibition and habituation of this reflex. Additionally, psychedelics are thought to induce neuroplastic changes, influencing synaptic plasticity and connectivity in the brain. This may contribute to the reported therapeutic effects of psychedelics, particularly in the context of mental health conditions. The neuroplastic changes can be quantified using testing paradigms that probe learning and memory formation, including eyeblink conditioning.

The neurological conditions may be a condition caused by, e.g., a neurological disorder, such as attention deficit hyperactivity disorder (ADHD). The neurological conditions may be a condition caused by, e.g., a chronic neurological disorder, such as narcolepsy.

In some embodiments, a method for identifying correct dosing in patients being treated with a medication for neurodeviate conditions may be provided. Referring to FIG. 1, the method 100 may include performing 110 a series of startle response tests of a user after the user has been administered a medication.

The series of tests will typically include three or more tests, each test occurring at a different time after the user has been administered the medication. The timing of the tests may vary. In some embodiments, the tests are performed hourly. In some embodiments, the pharmacokinetics and/or pharmacodynamics of the medication and person may determine the number of tests and when the tests are performed. In some embodiments, the time between a first test and a second test may be different than a time between the second test and a third test. In some embodiments, the time between each test may be equal.

Each test will typically be performed by a system of components.

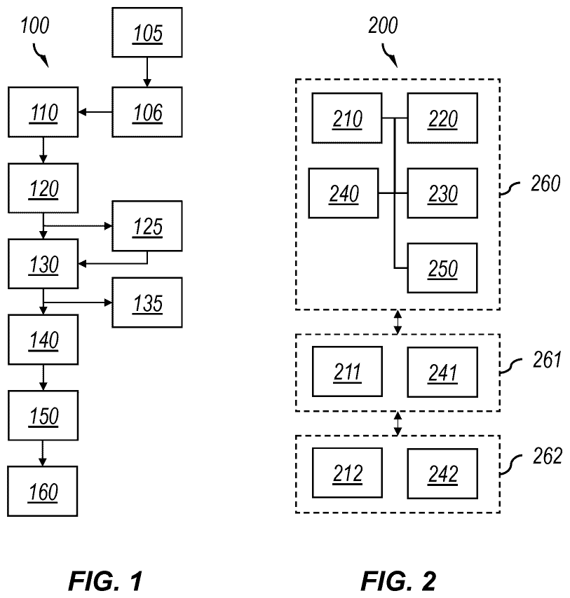

Referring to FIG. 2, in some embodiments, a system 200 may include one or more processors, which may include a first processor 210, a second processor 211, and/or a third processor 212.

As used herein, the term “processor” may include any combination of hardware, firmware, and software, employed to process data or digital signals. Processor hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs). In a processor, as used herein, each function may be performed either by hardware configured, i.e., hard-wired, to perform that function, or by more general-purpose hardware, such as a CPU, configured to execute instructions stored in a non-transitory storage medium. A processor may be fabricated on a single printed wiring board (PWB) or distributed over several interconnected PWBs. A processor may contain multiple processing units; for example a processor may include two processing units, an FPGA and a CPU, interconnected on a PWB.

In some embodiments, a display 220 may be operably coupled to the first processor. In some embodiments, a camera 230 may be operably coupled to the first processor. In some embodiments, a speaker 250 may be operably coupled to the first processor. In some embodiments, a non-transitory computer-readable medium 240 may be operably coupled to the first processor. In some embodiments, a non-transitory computer-readable medium 240, 241, 242 may be operably coupled to a respective processor (e.g., first processor 210, second processor 211, third processor 212, etc.). In some embodiments, each processor may be positioned within a separate housing. In some embodiments, the first housing 260 is a mobile device, such as mobile phone, tablet, or laptop. In some embodiments, the second housing 261 and/or the third housing 262 may be a remote server and/or a computing device associated with a psychopharmacologist or other medical professional. If multiple processors are used, the first processor may communicate with the second processor and/or the third processor.

The startle response may be tested using the camera, display, and/or speakers in any appropriate manner as understood by one of skill in the art. For example, at least one approach for performing a test of a startle response using a processor, camera, and a speaker as disclosed herein is described in greater detail in PCT/US2021/058698. This may include, e.g., using speakers to output white noise at a sufficient power level to evoke the startle response. The speaker may be, e.g., a speaker in a headphone or earphone. This may include having a blank screen change from a black background to a bright white background.

Referring to FIG. 1, in some embodiments, the method may include receiving 120 a plurality of images of at least one eye of the user from a camera during each test.

In some embodiments, the method may include calculating 130 amplitudes of a closure of an eyelid of at least one eye for each test, using the plurality of images. In some embodiments, this may be done by the first processor. In some embodiments, the plurality of images may be sent 125 to a remote processor (e.g., second processor 211, on a remote server, which may be within second housing 261), and the second processor will perform this step.

This step will typically involve various image processing steps to estimate how open or closed an eyelid is in any given image. Various techniques for accomplishing this are well-known in the art. A non-limiting example of how this can be done is can be understood as follows:

Computer vision and image processing techniques may be used to detect fully automated and real-time landmarks on a human face. More preferably, the algorithm is optimized to provide fast and accurate tracking of eyelids in both adults and infants. Any appropriate technique known to train a machine-learning algorithm can be utilized here.

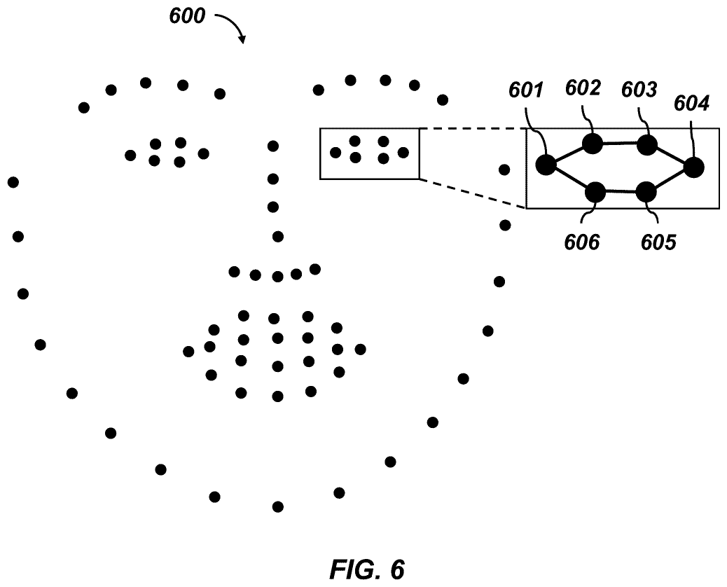

An algorithm may be used to detect a plurality of landmarks on the face. In FIG. 6, an example of a template 600, using 68 landmarks, is shown. In some embodiments, the template 600 may comprise or consist of 6 landmarks for each eye captured in the image. The six landmarks are, as seen in FIG. 6, a left corner 601, an upper left eyelid mark 602, an upper right eyelid mark 603, a right corner 604, a bottom right eyelid mark 605, and a bottom left eyelid mark 606.

Once the landmarks are identified, calculations can be made. Specifically, for each image, a Fraction Eyelid Closure (FEC) can be calculated. Using the preferred six landmarks as an example, conceptually, the calculation is made by looking at the differences in position of the six marks, and in particular:

$$FEC = \frac{( \begin{matrix}‖ Upper Left 602 - Lower Left 606 ‖ + \\ ‖ Upper Right 603 - Lower Right 605 ‖\end{matrix} )}{2 \times ( ‖ Left Corner 601 - Right Corner 604 ‖ )} \qquad ( 1 )$$

When looking at multiple images of the same individual, a normalized (“FEC_{NORM}”) can be determined, based on the minimum FEC (“FEC_{MIN}”) and maximum FEC (“FEC_{MAX}”). Specifically, FEC_{NORM}=1−(FEC−FEC_{MIN})/(FEC_{MAX}). An FEC_{NORM} of 0 corresponds to an eye that is fully open, and an FEC_{NORM} of 1 corresponds to an eye that is fully open.

In some embodiments, where two eyes are detected, various techniques may be used. An FEC may be calculated for each eye and the results may be, e.g., averaged together (or otherwise statistically combined). An FEC may be calculated for each eye, and the minimum value may be utilized. An FEC may be calculated for each eye, and the maximum value may be utilized. An FEC may be calculated for each eye, and a difference between the two FEC values may be determined. If the difference is above a threshold, the value of a flag may be set to 1 or a variable may be increased, indicating an anomalous response occurred.

In some embodiments, if no eyes are detected in a given image, or more than two eyes are detected, the image may be skipped.

A calibration sequence may have occurred prior to these steps, and FEC_{MIN} and FEC_{MAX} values may be determined based the images or video captured during calibration. In some embodiments, FEC_{MIN} and FEC_{MAX} values may be determined based solely on the images or video captured as part of the testing described above.

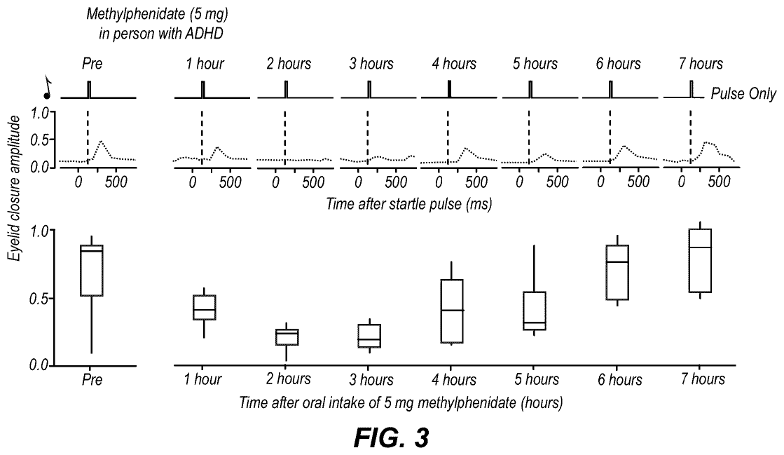

A schematic of the first steps in the method can be seen in FIG. 3.

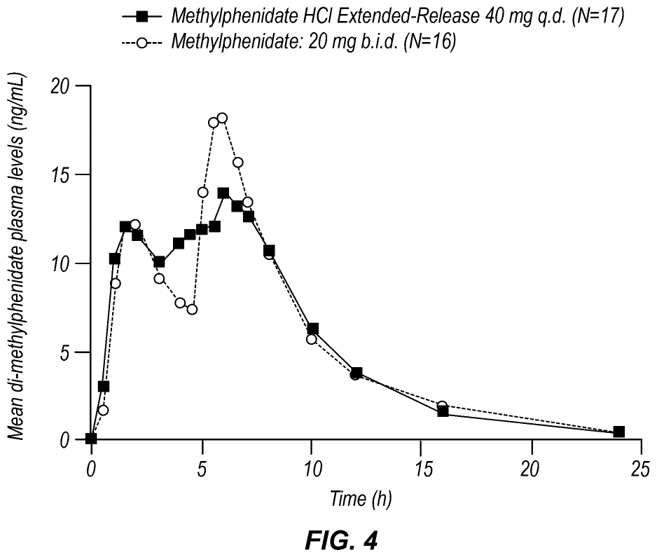

For each medication, a predetermined target plasma concentration curve may be stored in some fashion, e.g., in a database on a non-transitory computer-readable storage medium. The curve may be a table of data, or may be a curve such as the one shown in FIG. 4.

Once the tests have been completed, the method may include determining 140 a value of a correlation between a target plasma concentration curve of the medication and the amplitudes of the closure of the eyelid at the different times after the user has been administered the medication.

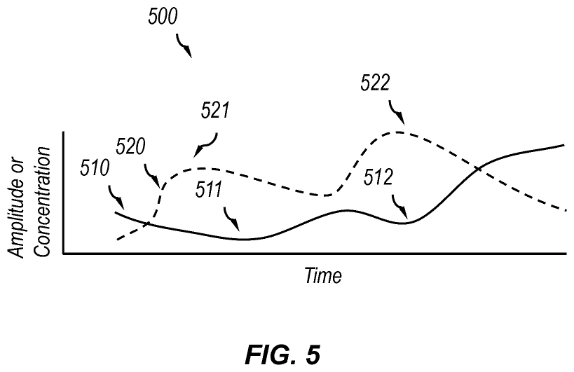

In some embodiments, the shape of the target plasma concentration curve is compared to the shape of a curve that has been fit to the calculated amplitudes over time. Referring to FIG. 5, in an example comparison 500, a first curve 510 based on the amplitude data in FIG. 3 is compared on arbitrary axes to a second curve 520 of one of the plasma methylphenidate concentration curves from FIG. 4, it can be seen that the first curve has a first inverted peak 511 and a second inverted peak 512 that is generally aligned with a first peak 521 and a second peak 522, respectively, of the second curve. Further, it can be seen that, for this medication, the amplitude curve (first curve 510) is negatively correlated with the plasma methylphenidate concentration curve—that is, when the plasma concentration curve shows a maximum concentration, the eyeblink amplitude shows a minimum concentration. In some embodiments, the amplitude is positively correlated with the plasma concentrations of the medication. In some embodiments, the amplitude is negatively correlated with the plasma concentrations of the medication.

In some embodiments, the plasma concentrations may be normalized before being compared to the amplitude data. In some embodiments, the plasma concentrations may be stored as normalized data, such that the system does not need to normalize the plasma concentrations to determine a value of a correlation.

In some embodiments, the amplitude data may be inverted or otherwise modified to make a correlation or comparison easier. For example, in some embodiments, the y-axis values of the curve are determined by mAⁿ, where A is the determined amplitude at a given point, m is a weighting factor (e.g., a value 0-1), and n is 1 or −1.

In some embodiments, a value can be assigned based on the similarity of the two Such similarity measurements can be determined using known techniques, such as via curves. Fréchet distances, root-mean-square differences, etc.

In some embodiments, a value can be assigned based on a least-squares fit of the amplitudes (or the modified amplitudes) to the plasma medication concentrations.

In some embodiments, curves are not compared; rather, an amplitude at a time point T after being medicated is compared to a concentration determined by interpolating tabulated plasma medication concentration data.

In some embodiments, the method may include determining 150 whether a correct or sufficient dose has been achieved based on the value of the correlation. In some embodiments, this may be done by comparing the value of the correlation to a threshold. The determining may include notifying an individual (such as the user, a doctor, etc.) if a correct or sufficient dose was achieved.

In some embodiments, all steps are performed on the device, such as a mobile device, that includes the camera used to capture the plurality of images—referring to FIG. 2, all steps may be performed on the device associated with the first housing 260.

In some embodiments, a first device (such as a mobile device) sends the plurality of images to a remote processor (such as second processor 211 in FIG. 2), where the remote processor is configured to perform the calculating 130, determining 140 of correlations, and determining 150 of whether a sufficient dose has been achieved.

In some embodiments, the method may include recommending 160 (or generating a recommendation of) a modified dosage of the medication based on the correlation. For example, in some embodiments, for a given medication, if a correlation value is in a first range, it may indicate a relatively small change to the dose is appropriate, while if the correlation value is in a second (lower) range, it may indicate a relatively larger change to the dose is appropriate.

Additionally, in some embodiments, the amplitudes or statistics related to the amplitudes may be used to determine whether the dosage should be increased or decreased. For example, if the standard deviation of the amplitudes are in a first (e.g., high) range, it may indicate the dosage should be increased, while if the standard deviation of the amplitudes are in a second (e.g., low) range, it may indicate the dosage should be increased.

In some embodiments, the method may include repeating the steps at the same medication dosage. In some embodiments, the method may include adjusting a dosage of the medication and repeating the steps.

In some embodiments, the method may include receiving 106 input indicating when the medication was administered. For example, in some embodiments, the person being treated may enter this information using an input device (such as a keyboard, etc.). In some embodiments, a psychopharmacologist or other medical professional may enter this information. This information is typically then sent to the one or more processors.

In some embodiments, the method may include receiving 105 input indicating what dosage was administered. For example, in some embodiments, the person being treated may enter this information using an input device (such as a keyboard, etc.). In some embodiments, a psychopharmacologist or other medical professional may enter this information. In some embodiments, this may be done via one or more pieces of equipment configured to administer the medication. For example, an automated injector may have a processor configured to cause the dispensation of a fixed amount of medication into a user (e.g., intravenously), then transmit that information to the one or more processors automatically.

In some embodiments, the method may include storing 135 information in a database, the information including a user code, a dose, a time of administration, and the amplitudes and times each of the plurality of images was captured. This step may occur at any time in the process after the relevant information is gathered, and/or may occur over multiple times (for example, dose and time of administration may be stored before any startle test is performed, while the times that each image was captured may be stored immediately after (or in parallel with) the images being captured).

Referring to FIG. 2, the system 200 includes a non-transitory computer-readable medium 240, 241, 242 containing instructions that, when executed, configure the one or more processors to perform the method as disclosed herein.

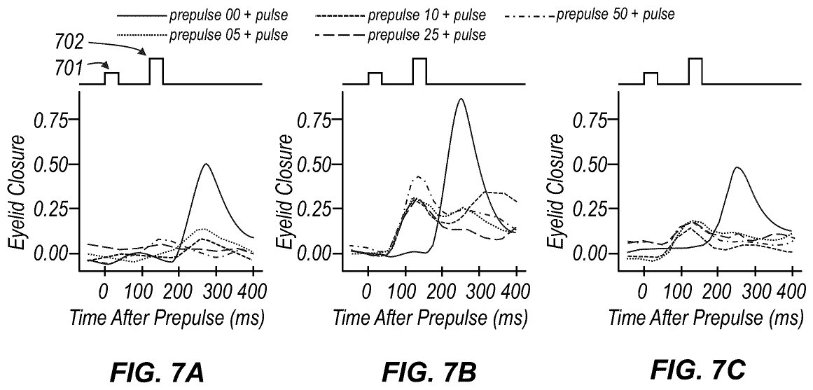

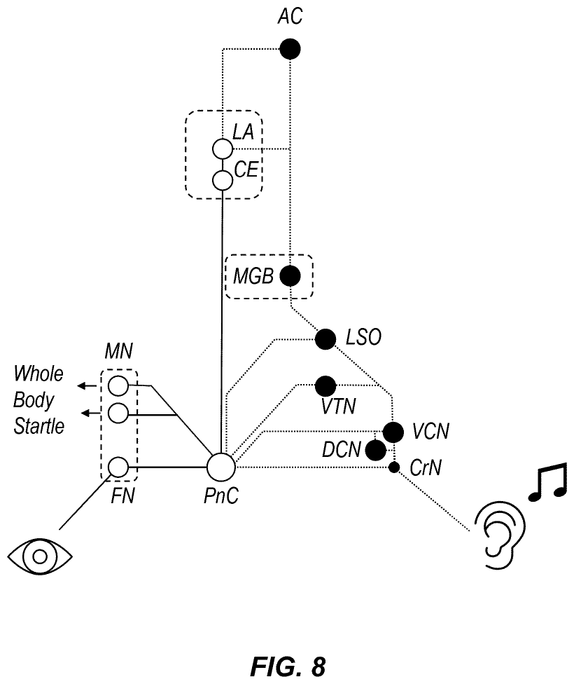

Referring to FIGS. 7A-7C, various tests were performed relating to prepulse inhibition (PPI). PPI is the behavioral phenomenon whereby the magnitude of the startle response is inhibited when a short and loud startling sound (the pulse) is preceded by a weaker sound that does not elicit a startle reflex (the prepulse). Herewith, PPI measures sensorimotor gating, which is the mechanism of the nervous system to filter out irrelevant sensory information to protect the brain from overstimulation and enabling appropriate reaction to stimuli that are relevant. PPI is less brain region specific and probes midbrain function and modulatory effects that the midbrain receives from limbic systems, thalamus, and prefrontal areas.

The method generally comprises several steps. To test prepulse inhibition, the method optionally begins by first emitting a white noise prepulse, the white noise prepulse 701 having a first strength configured to not elicit a startle reflex in the user. The lack of a startle reflex following this prepulse can optionally be confirmed by capturing one or more images after the prepulse is emitted, and not detecting any substantial degree of eyelid closure as described above with respect to eyeblink conditioning.

After a delay, the method may then include emitting a white noise pulse 702 having a second strength configured to elicit a startle reflex in the user, the second strength being greater than the first strength. The existence of a startle reflex following this pulse can optionally be confirmed by capturing one or more images after the pulse is emitted, and determining a first degree of eyelid closure.

As seen in FIG. 7A, average responses of nine individuals considered to be neurotypical is seen. In comparison, the average responses of ten individuals diagnosed with ADHD, being treated with methylphenidate, can be seen before (FIG. 7B) and after (FIG. 7C) treatment. As seen, prior to treatment, the degree of eyelid closure is dramatically larger for every intensity of the prepulse (00, 05, 10, 25, and 50) as compared to the neurotypical behavior. In FIGS. 7A-7C, “prepulse XX” indicates that the prepulse was presented at about XX % of the pulse intensity (e.g., “prepulse 25” indicates the prepulse was presented at about 25% of the pulse intensity). Surprisingly, a dramatic reduction in eyelid closure for every intensity of the prepulse can be seen after medicating with methylphenidate, to levels similar to those of neurotypical responses.

In FIG. 8, neurocircuitries underlying auditory startle reflexes can be seen. Some elements of the auditory system (solid lines, solid circles) and efferents (dotted lines and open circles) are shown. The fastest route for transmission of acoustic input into motor output is from the CrN via the PnC to the motor neurons, including the FN. In addition, multiple afferent systems including the LSO, VTN, DCN, and VCN excite the giant PnC neurons. Amygdala activity directly controls the expression of the startle reflex by its projections to the PnC. Thus, it is expected that anything that modulates the efferents involved here, or influences the pathways of the startle reflexes, can be detected.

While the invention is described through the above-described exemplary embodiments, modifications to, and variations of, the illustrated embodiments may be made without departing from the inventive concepts disclosed herein. For example, although specific parameter values, such as dimensions and materials, may be recited in relation to disclosed embodiments, within the scope of the invention, the values of all parameters may vary over wide ranges to suit different applications.

As used herein, including in the claims, the term “and/or,” used in connection with a list of items, means one or more of the items in the list, i.e., at least one of the items in the list, but not necessarily all the items in the list.

Disclosed aspects, or portions thereof, may be combined in ways not listed above and/or not explicitly claimed. In addition, embodiments disclosed herein may be suitably practiced, absent any element that is not specifically disclosed herein. Various modifications may be made to the systems, methods, apparatus, mechanisms, techniques and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined of the claims.

1. A method for identifying correct dosing in patients being treated with a medication for neurodeviate conditions, the method comprising:
performing three of more tests of a startle response of a user, each test utilizing a mobile device having a camera, display, and optionally a speaker, each test occurring a different time after the user has been administered a medication;
receiving a plurality of images of at least one eye of the user from a camera during each test;
calculating one or more amplitudes of a closure of an eyelid of the at least one eye for each test;
determining a value of a correlation between predetermined plasma concentrations of the medication and the one or more amplitudes of the closure of the eyelid at the different time of each test after the user has been administered the medication; and
determining whether a correct dose has been achieved based on the value of the correlation.

2. The method of claim 1, wherein all steps are performed on the mobile device.

3. The method of claim 1, wherein the mobile device sends the plurality of images to a remote processor, the remote processor being configured to calculate the one or more amplitudes, determine the correlation, and determining whether the correct dose has been achieved.

4. The method of claim 1, wherein the one or more amplitudes are positively correlated with a plasma concentrations of the medication.

5. The method of claim 1, wherein the one or more amplitudes are negatively correlated with a plasma concentrations of the medication.

6. The method of claim 1, further comprising recommending a modified dosage of the medication based on the correlation.

7. The method of claim 1, further comprising repeating, at a same medication dosage, steps of performing tests of a startle response, receiving images, calculating amplitudes, determining values of correlations, and determining whether the correct dose has been achieved.

8. The method of claim 1, further comprising adjusting a dosage of the medication and repeating steps of performing tests of a startle response, receiving images, calculating amplitudes, determining values of correlations, and determining whether the correct dose has been achieved.

9. The method of claim 1, further comprising receiving input indicating when the medication was administered.

10. The method of claim 9, further comprising receiving input indicating what dosage was administered.

11. The method of claim 1, further comprising storing information in a database, the information including a user code, a dose, a time of administration, and the one or more amplitudes and times each of the plurality of images was captured.

12. A system, comprising:
one or more processors;
a display operably coupled to a first processor of the one or more processors;
a camera operably coupled to the first processor;
optionally a speaker coupled to the first processor; and
a non-transitory computer-readable medium containing instructions that, when executed, configure the one or more processors to:
perform three of more tests of a startle response of a user, each test utilizing the display, the speaker, or both, and each test occurring a different time after the user has been administered a medication;
receive a plurality of images of at least one eye of the user from the camera during each test;
calculate one or more amplitudes of a closure of an eyelid of the at least one eye for each test; and
determine a correlation between predetermined plasma concentrations of the medication with a curve formed by the one or more amplitudes of the closure of the eyelid at different times after the user has been administered the medication.

13. The system of claim 12, wherein the instructions, when executed, further configure the one or more processors to determine whether a correct dose has been achieved based on a value of the correlation.

14. The system of claim 12, wherein the first processor is present on a mobile device and all steps are performed on the mobile device.

15. The system of claim 12, wherein the first processor is present on a mobile device, a second processor of the one or more processors is present on a remote device, the first processor is configured to send the plurality of images to the second processor, the second processor being configured to calculate the one or more amplitudes, determine the correlation, and determining whether a correct dose has been achieved.

16. The system of claim 12, wherein the one or more amplitudes is positively correlated with a plasma concentrations of the medication.

17. The system of claim 12, wherein the one or more amplitudes is negatively correlated with a plasma concentrations of the medication.

18. The system of claim 12, wherein the instructions, when executed, further configure the one or more processors to recommend a modified dosage of the medication based on the correlation.

19. The system of claim 12, wherein the instructions, when executed, further configure the one or more processors to receive input indicating when the medication was administered.

20. The system of claim 19, wherein the instructions, when executed, further configure the one or more processors to receive input indicating what dosage was administered.

21. The system of claim 12, wherein the instructions, when executed, further configure the one or more processors to store information in a database, the information including a user code, a dose, a time of administration, and the one or more amplitudes and times each of the plurality of images was captured.
